(19) United States
(12) Patent Application Publication
(10) Pub. No.: US 2008/0269579 A1
(43) Pub. Date: Oct. 30, 2008

(54) System for Monitoring Changes in an Environmental Condition of a Wearer of a Removable Apparatus

(76) Inventor: Mark Schiebler, Maitland, FL (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/741,882

(22) Filed: Apr. 30, 2007

(51) Int. Cl. A61B 5/053; A61B 5/00; G01P 15/00

(52) U.S. Cl. 600/323; 600/300; 600/547; 73/488

(57) ABSTRACT

A system for detecting at least one of an environmental condition and a physical condition of a user, the system including a removable apparatus that may be placed in contact with the user when a change in at least one environmental condition is expected, a probe as part of the removable apparatus for detecting the change in the at least one environmental condition, a power source as part of the removable apparatus for powering the probe, a monitoring unit, and a communication system for communicating between the removable apparatus and the remote monitoring unit.

### FIELD OF THE INVENTION

This invention relates generally to an apparatus for detecting and reporting environmental conditions of a wearer and, more particularly, to a mouthpiece and/or patch which may be used to determine changes in an environmental condition unique to the wearer.

### BACKGROUND OF THE INVENTION

Current mouthpieces, such as athletic mouth guards, are usually formed from thermoplastic materials and come in a variety of shapes, designs, thicknesses, and sizes. Each is designed to protect a user's teeth against physical shock or blows either directly to the teeth, from the teeth themselves, and/or against a user's jaws, head, or even the user's body. Such mouthpieces are also designed to ensure the mouthpiece stays in position and prevent bite through of the mouthpiece by the user either during normal use or upon the application of shock or blows. Such mouthpieces also may absorb, attenuate, or deflect such blows to decrease the resultant transmitted force in an attempt to decrease or minimize injury to the user.

A myriad of designs exist which attempt to reduce such injury. Such protection has been afforded by custom fitting and/or by the inclusion of ribs, embossing, chambers, inserts, devices, or by simply increasing the thickness of the mouthpiece thereby increasing its bulk. This increased bulk may also increase tongue and breathing interference with resultant discomfort to the user. Some such mouthpieces are composite designs which increase production costs and may lead to higher failure rates.

The sort of injury that the myriad of designs of mouth pieces are to prevent generally pertain to injuries that might be caused by teeth coming together as a result of a jar, tight jaw clench (“locked jaw”), or a blow. For example, activities where mouthpieces are usually worn, participants may become injured as a result of blows or jars received while engaged in such activities. Mouthpieces are used to prevent, or at a minimum, reduce injuries to the teeth from occurring.

When used for protection when a wearer participates in sporting events, a wearer may encounter other injuries that may be preventable if information about the wearer is known in advance of a wearer becoming afflicted by the prospective injury. For example, for outside activities, heat exhaustion has been known to lead to death. Whereas in some contact sports, such as but not limited to boxing, rugby, and football, an impact to a wearer's head may be a precursor to a more serious injury if the wearer is again hit in the head region. (e.g., athletic post concussive syndrome and conditions associated with being punch drunk).

Individuals and entities involved in such activities would benefit from a mouthpiece and/or the wearable device that is able to provide additional information about the wearer to prevent a future injury, rather than just preventing injuries that might be caused by teeth coming together as a result of a jar or a blow.

### BRIEF DESCRIPTION OF THE INVENTION

Exemplary embodiments of the invention disclose a system, method and computer software code for determining a change in an environmental condition unique to the wearer. A system for detecting at least one of an environmental condition and a physical condition of a user is disclosed. The system includes a removable apparatus that may be placed in contact with the user when a change in an environmental condition is expected. A probe is also provided as part of the removable apparatus for configured to detect the change in the environmental condition. A power source is also included as part of the removable apparatus configured to power the probe. A monitoring unit is provided. A communication system is further included which is configured for communicating between the removable apparatus and the remote monitoring unit.

In another embodiment a method for detecting at least one of an environmental condition and a physical condition of an entity active in a competitive event wherein a removable apparatus with a probe to detect a change in the environment condition unique to the entity is attached to the entity is disclosed. The method includes a step for detecting a change in environmental condition at the removable apparatus. A step for monitoring the change in environmental condition is also provided. A step for reporting the change in environmental condition so that the individual may be further evaluated is further disclosed.

In yet another exemplary embodiment, a computer software code implemented through at least one processor for detecting at least one of an environmental condition and a physical condition of an entity active in a competitive event wherein a removable apparatus with a probe to detect a change in the environment condition unique to the entity is attached to the entity is disclosed. The computer software code includes a computer software module for detecting a change in the environmental condition at the removable apparatus, and a computer software module for monitoring the change in environmental condition. A computer software module for reporting the change in environmental condition so that the individual may be further evaluated is also disclosed.

### BRIEF DESCRIPTION OF THE DRAWINGS

A more particular description of the invention briefly described above will be rendered by reference to specific embodiments thereof that are illustrated in the appended drawings. Understanding that these drawings depict only typical embodiments of the invention and are not therefore to be considered to be limiting of its scope, exemplary embodiments of the invention will be described and explained with additional specificity and detail through the use of the accompanying drawings in which:

FIG. 1 depicts an exemplary illustration of mouthpiece for determining a variety of environmental and physical conditions of a wearer;

FIG. 2 depicts an exemplary illustration of a removable patch for determining a wearer's temperature and/or impact acceleration/deceleration;

FIG. 3 depict an exemplary illustration of locations upon a human body where embodiments of the present invention may be located;

FIG. 4 depicts an exemplary illustration of a location upon an animal, such as a horse, where an embodiment of the present invention may be located;

FIG. 5 depicts a simplified exemplary block diagram of a system for monitoring a mouthpiece wearer's temperature and/or impact acceleration/deceleration; and

FIG. 6 depicts a flowchart of steps of an exemplary embodiment of the present invention.

### DETAILED DESCRIPTION OF THE INVENTION

Reference will now be made in detail to the embodiments consistent with the invention, examples of which are illustrated in the accompanying drawings. Wherever possible, the same reference numerals used throughout the drawings refer to the same or like parts.

Though exemplary embodiments of the present invention are described with respect to a mouthpiece worn during activities that may involve contact, such as contact sports and other competitive activities. The competitive activities are not limited to just when a wearer is competing. Competitive activities are also meant to cover times when the wearer is training and/or practicing. Exemplary embodiments of the invention are also applicable for other uses, such as but not limited to mouthpieces used for medical reasons, such as when individuals may experience seizures. Furthermore, though mouthpieces are primarily disclosed, the elements of the invention may also be used in other devices that might be worn and/or attached to a user during a specific activity, for example, a patch, such as one applied directly to an individual's body, either directly and/or as part of a garment.

Exemplary embodiments of the invention solves the problems in the art by providing a system, method, and computer implemented method, such as a computer software code for determining environmental conditions being experienced by an individual. Persons skilled in the art will recognize that an apparatus, such as a data processing system, including a CPU, memory, I/O, program storage, a connecting bus, and other appropriate components, could be programmed or otherwise designed to facilitate the practice of the method of the invention. Such a system would include appropriate program means for executing the method of the invention.

Also, an article of manufacture, such as a pre-recorded disk or other similar computer program product, for use with a data processing system, could include a storage medium and program means recorded thereon for directing the data processing system to facilitate the practice of the method of the invention. Such apparatus and articles of manufacture also fall within the spirit and scope of the invention.

Broadly speaking, the technical effect is a device to determine environmental conditions being experienced by an individual. To facilitate an understanding of the exemplary embodiments of the invention, it is described hereinafter with reference to specific implementations thereof. Exemplary embodiments of the invention may be described in the general context of computer-executable instructions, such as program modules, being executed by a computer. Generally, program modules include routines, programs, objects, components, data structures, etc. that perform particular tasks or implement particular abstract data types. For example, the software programs that underlie exemplary embodiments of the invention can be coded in different languages, for use with different platforms. In the description that follows, examples of the invention may be described in the context of a web portal that employs a web browser. It will be appreciated, however, that the principles that underlie exemplary embodiments of the invention can be implemented with other types of computer software technologies as well.

The data of individual players or users can be combined in a film review of the game or event to produce a team dynamic of physical performance under practice or game conditions. Also this data can be converted over generating the performance criteria for commercial game simulations using current computer gaming programs for each sport, such as but not limited to EA sports Madden Football™.

Moreover, those skilled in the art will appreciate that exemplary embodiments of the invention may be practiced with other computer system configurations, including hand-held devices, multiprocessor systems, microprocessor-based or programmable consumer electronics, minicomputers, mainframe computers, and the like. Exemplary embodiments of the invention may also be practiced in distributed computing environments where tasks are performed by remote processing devices that are linked through a communications network. In a distributed computing environment, program modules may be located in both local and remote computer storage media including memory storage devices.

Referring now to the drawings, embodiments of the present invention will be described. Exemplary embodiments of the invention can be implemented in numerous ways, including as a system (including a computer processing system), a method (including a computerized method), an apparatus, a computer readable medium, a computer program product, a graphical user interface, including a web portal, or a data structure tangibly fixed in a computer readable memory. Several embodiments of the invention are discussed below.

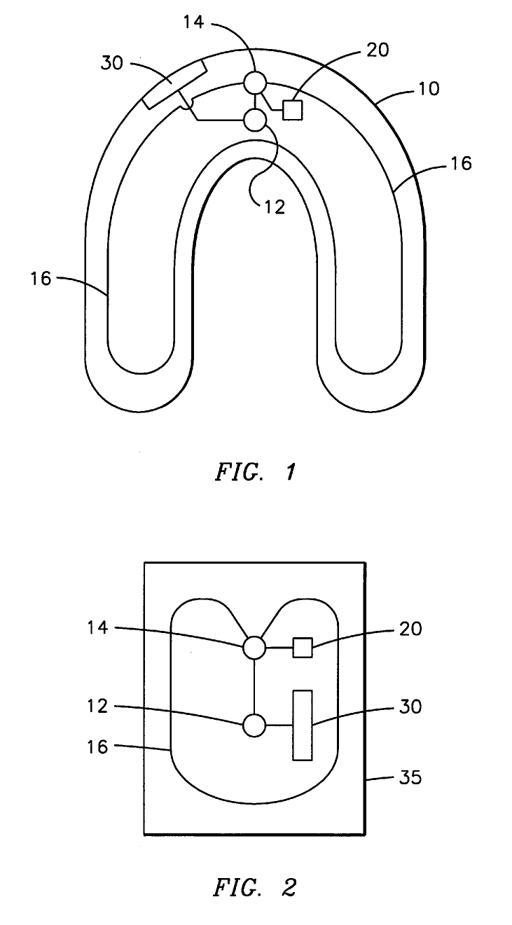

FIG. 1 depicts an exemplary illustration of a mouthpiece for determining a variety of environmental conditions and physical conditions of a wearer. The mouthpieces 10 may include any and all elements that are usually included in a mouthpiece to prevent damage to teeth. A probe 12 is included as part of the mouthpiece 10 for detecting a change in an environmental condition and physical conditions. Such conditions include, but are not limited to the wearer's temperature, impact deceleration, acceleration, location, velocity, sweat composition, saliva composition, ratio of oxyhemoglobin to deoxyhemoglobin, heart rate. Oxy/deoxy hemoglobin saturation ratio may be measured by use of an infrared spectroscopy device within the probe 12. Measuring the oxy/deoxy hemoglobin saturation ratio can show that the wearer is beyond his/her usual aerobic threshold for exercise, and how fast recovery occurs. Likewise, the probe 12 may include the appropriate measuring device for the specific environmental condition and/or physical condition to be measured.

For example, another environmental condition that may be evaluated is measuring a wearer's sweating ability. The probe may be a skin impedance/conductance measurer. As a wearer's dehydrates, the impedance/conductance of the skin is also likely to change since the sweat composition would also change. Skin chloride or sodium could be monitored as well.

The probe 12 may have a plurality of embodiments, such as but not limited to an embedded computer chip and/or microprocessor. With respect to determining temperature, the probe may be a temperature probe. With respect to determining velocity change, either acceleration and/or deceleration, the probe may include an accelerometer. A chip may be further included to compare readings to an acceptable reading for each type of measurement taken. Though various environmental conditions specific to a wearer are discussed above, a plurality of these environmental conditions may be evaluated with a single mouthpiece 10 wherein the mouthpiece has a probe 12 able to analyze all conditions desired.

A power source 14 is also provided to power to the probe 12. The power source 14 may have a plurality of embodiments. As illustrated the power source 14 includes wire loop 16 to allow for inductive charging of the probe 12 and/or a rechargeable power source 14 connected to the probe 12. In another exemplary embodiment a receiver unit (not shown) is provided on the mouthpiece 10 to allow the mouthpiece 10 to be attached to, or plugged into, a remote charging unit (not shown).

A monitoring unit 20 is also provided. In an exemplary embodiment when remote from the mouthpiece, the monitoring unit 20 is a computer. The monitoring unit 20 may also be part of the mouthpiece 10. When part of the mouthpiece 10, the monitoring unit 20 may be a microprocessor. When a wearer exceeds a limit the probe 12 is monitoring, the information is reported to the monitoring unit 20. The monitoring unit 20 may also include a storage device 22 to maintain probe readouts. In another exemplary embodiment a second remote unit 22 is also used. The second remote unit 23 may receive probe readings for uses in other endeavors wherein the storage device 22 is part of the second unit.

Should the probe 12, power source 14, and/or monitoring unit 20 fail, each element is removable. A replacement element 12, 14, 20 may then be inserted. Thus, the connections between these elements 12, 14, 20 are fixed within the mouthpiece 10. Likewise, these elements 12, 14, 20 are also replaceable in the patch 35 disclosed herein.

For example, with respects to sports that computer simulation games are made to emulate, the information may be provided for game simulation games wherein how a respective wearer performs over a course of the sporting event is taken into account performance versus the change in the wearer's unique environmental conditions. Similarly, such information may be communicated from a game simulation program to the remote monitoring unit 20 for monitoring so that the remote monitoring unit 20 can compare current wearer's data against prior data. The prior data that current data may be compared to is not limited to data about a particular wearer. For example, the prior data may be data gathered about other entities that have played the same position as the wearer currently being monitored. In another exemplary example, the prior data may be data gathered about entities experiencing similar environmental conditions, such as but not limited to extreme external heat or cold conditions which may or may not be based on the same field of play and/or stadium, and then compared against the current wearer. This information may be relayed to a coach and/or trainer to further assist in making coaching/training decisions.

Though storage of information is described as occurring at the remote unit 20, 23, storage of probe information may also be accomplished within the mouthpiece 10. The information may then be downloaded a plurality of ways from the mouthpiece 10 to the remote monitoring 20. The download may be accomplished through a wireless download system and/or a wired download system.

FIG. 2 depicts an exemplary illustration of a removable patch 35 for determining a wearer's temperature and/or impact acceleration/deceleration. All features and/or embodiments described above with respect to the mouthpiece 10 may also be used for the patch 35. The term patch is not meant to be limiting. The patch 35 may be an adhesive 14 applied element. The patch 35 may also be integrated into a piece of sporting equipment, such as football shoulder pads, and/or an article of clothing, such as a uniform jersey. A grouping of patches may be used on a single wearer. The grouping of patches is not limited only to patches. A mouthpiece may be along with a grouping of patches. In an exemplary embodiment, a grouping of patches may allow for recording of an electrocardiogram during use.

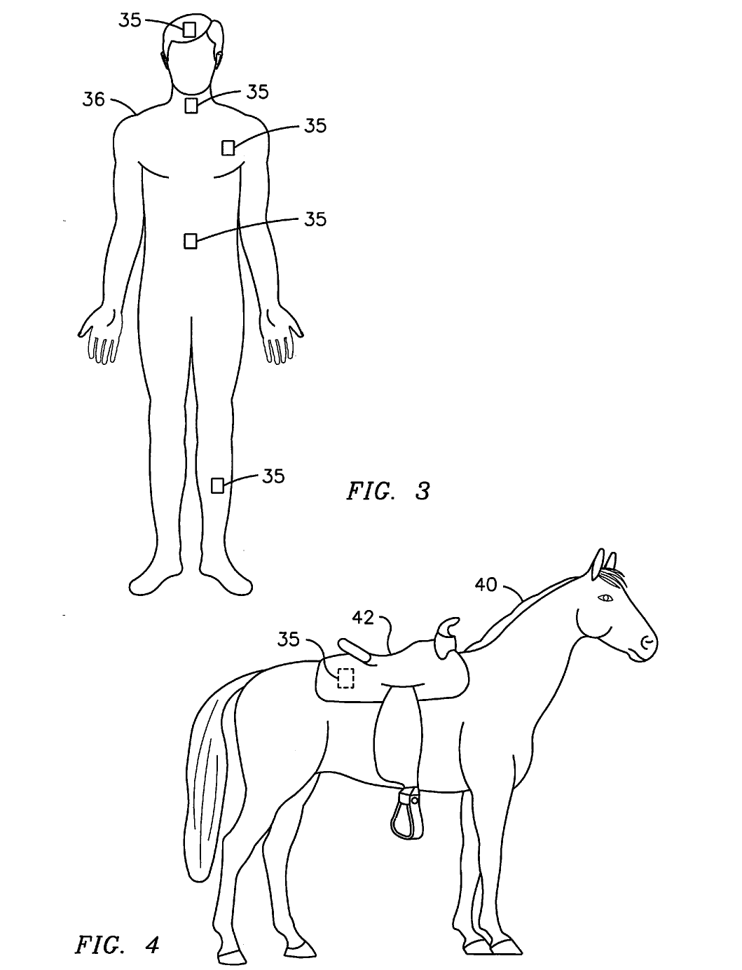

Furthermore, exemplary embodiments of the present invention may be used by more than just humans. FIG. 3 depicts an exemplary illustration of locations upon a human body 36 where embodiments of the present invention may be located. The locations are limitless. FIG. 4 depicts an exemplary illustration of a location upon an animal, such as a horse 40 where an embodiment of the present invention may be located. With respect to the horse, the patch 35 is making contact to the horse under a saddle 42, or saddle blanket.

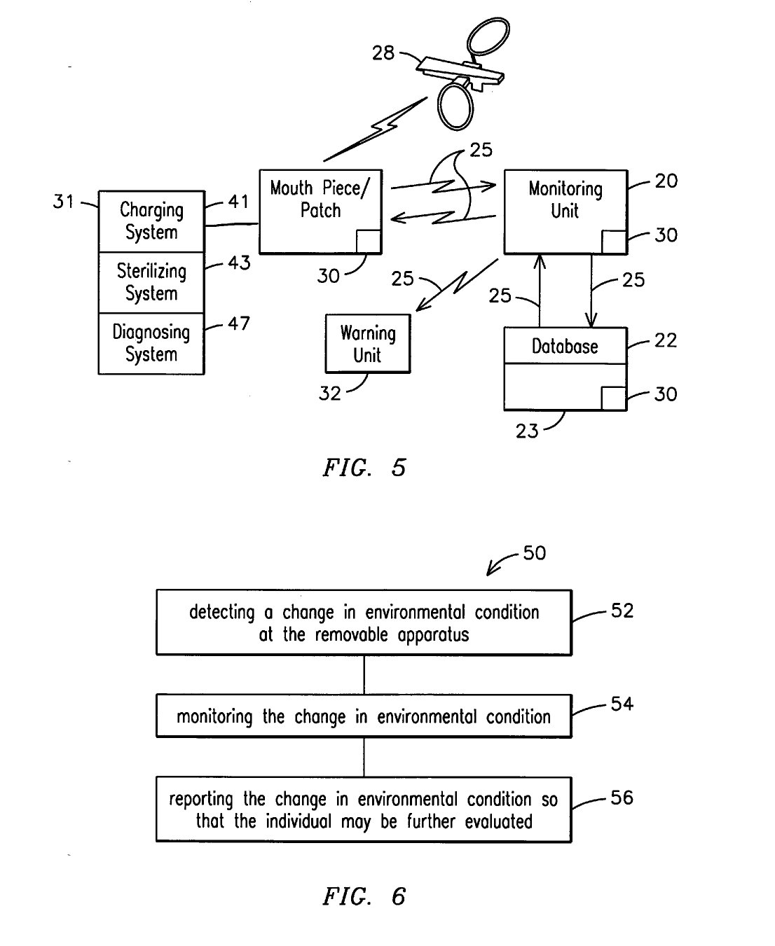

FIG. 5 depicts a simplified block diagram of an exemplary system for monitoring a mouthpiece 10 and/or patch 35 wearer's environmental condition changes. As illustrated a change in a particular environmental condition is communicated to a remote unit 20. When the environmental condition exceeds a specific limit a warning may be communicated to the mouthpiece 10 and/or patch 35 as well as the warning unit 32. Additionally data collected at the remote unit 20 may be transmitted to a processor 23 that collects the information for retention in a database 22. As further illustrated and discussed above, information from the database 22 may also be sent to the remote unit 20 to compare the wearer's environmental conditions to either that wearer's prior recorded data and data collected from others.

As further illustrated in FIG. 5, a communication system 25 for communicating between the removable apparatus 10 and the remote monitoring unit 20 is provided. The communication system is also provided for communicating between the remote unit 26 and the second remote unit 23. In an exemplary embodiment the communication system 25 may be a radio frequency identification (RFID) communication system. Other communication systems, such as but not limited to Bluetooth™ technology, may also be used provided that these systems may allow transmission of data, reception of data, and identification of where the data is coming from. Towards this end each element has appropriate transmitting and receiving components as part of the element. The communication system 25 must be able to accomplish its functions in nearly a simultaneous fashion since more than one mouthpiece 10 may be used over the same communication system 25 at the same time. Under such a system data, or information unique to each mouthpiece 10 is provided so that the remote unit 20, 23 may be used to read a plurality of data from multiple mouthpieces nearly simultaneously.

A tracking system 28, such as a global positioning-based system may also be linked to the mouthpiece 10. The tracking system 28 may be used to determine that a wearer is in a proper location. For example, with respects to a football team, an individual monitoring location of wearers may be able to using the tracking system 28 to determine whether the proper players and/or enough players are on the field at any given time.

A warning device 30 is also included. The warning device 30 provides notice when the change in the environmental condition exceeds a predetermined limit. The warning may either be an audible and/or visible warning. In an exemplary embodiment, the warning device 30 is part of the remote unit 20, 23. In another exemplary embodiment, the warning device 30 is an independent warning unit 32. In operation, the remote unit 20 may be positioned at a location that is safe from getting disrupted if nearer a field of play. The warning device 32 may then be used proximate the field of play, such as but not limited to along a side line.

In another exemplary embodiment, the warning device 30 is part of the mouthpiece 10. For example, if a wearer's temperature exceeds a specific limit, either all or a part of the mouthpiece 10 may turn a different color. Similarly, a miniature siren may be part of the mouthpiece 10 and emits a sound when the specific environmental condition is exceeded. In another exemplary embodiment, all three, or two, warning devices 30 disclosed herein may be utilized together.

A storage device 31 may also be provided as part of the system. In an embodiment, the storage device 31 has a charging system 41. For example, a power source is provided so that when the apparatus 10, 35 is in contact with the storage device 31 the power source 14 that is part of the removable apparatus 10, 35 is charged. In an embodiment, the storage device 31 has a sterilizing system 43 that is configured to sterilize the apparatus when connected. The storage device 31 may also include a diagnostic system 47 to determine whether the probe 12 is functioning properly, such as but not limited to insuring that the probe 12 is accurately measuring at least one of the environmental conditions that it is suppose to measure, when the apparatus 10, 35 is in contact. In operation, the apparatus 10, 35, such as but not limited to the mouthpiece 10 and/or patch 35, is placed in contact with the storage device 31 at the end of a daily use. The apparatus 10, 35 is then charged, sterilized, and/or diagnosed. Those skilled in the art will readily recognize that all of these functions may be performed each time the apparatus is in contact with the storage device or the storage device is mechanized so that the user is able to select only one of these functions to be performed. Furthermore, each of these systems 41, 43, 47 may be independent systems where their functions are performed remote from the apparatus 10, 35.

FIG. 6 depicts a flowchart of exemplary steps within an exemplary embodiment of the invention. As illustrated the flowchart 50 provides for a step for detecting a change in environmental condition at the removable apparatus, step 52. The change in environmental condition is monitored, step 54. Monitoring may occur at the mouthpiece and/or patch, and at a remote location. The change in environmental condition is reported so that the individual may be further evaluated, step 56. The report may be in the form of a warning alarm. As disclosed above, these steps may be implemented using a computer software code and/or algorithm implemented through at least one processor.

There has been recent news of repetitive athletic concussions and brain damage in boxers and professional football players. The autopsy of safety Ricky Watters, one of the more recent examples of this, showed evidence of advanced degeneration for age particularly in the frontal lobes of his brain. While the exact relationship between head trauma during sports events is an ongoing debate within the medical and sports community, the fact that the white-gray matter interface in the brain may undergo sheer injury with rapid deceleration is not. This is a well known phenomenon in neuro-imaging after motor vehicle accidents. These types of injuries may be severe enough to cause peticheal hemorrhages at gray-white matter junction. Microtears of the critical connections of the gray and white matter could also theoretically occur. The number and severity of “head hits” so to speak could be tracked for each player using an exemplary embodiment of the present invention and its linked storage media. Recording of this type of information could help in beginning to understand the causal relationship between head trauma in sports and premature brain damage.

While exemplary embodiment of the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. A system for detecting at least one of an environmental condition and a physical condition of a user, the system comprising:
a) a removable apparatus that may be placed in contact with the user when a change in the at least one environmental condition is expected;
b) a probe as part of the removable apparatus for detecting the change in the at least one environmental condition;
c) a power source as part of the removable apparatus for powering the probe;
d) a monitoring unit; and
e) a communication system for communicating between the removable apparatus and the remote monitoring unit.

2. The system according to claim 1, wherein the removable apparatus is a mouthpiece.

3. The system according to claim 1, wherein the removable apparatus is a patch that is attachable and removable to a part of the user's body wherein the patch does not interfere with an activity the user is participating in.

4. The system according to claim 1, wherein the removable apparatus is at least one of a piece of equipment and an article of clothing.

5. The system according to claim 1, wherein the probe comprises a computer chip.

6. The system according to claim 1, wherein the probe comprises a temperature probe.

7. The system according to claim 1, wherein the probe comprises a velocity probe to detect at least one of acceleration, deceleration, and a rate of change thereof.

8. The system according to claim 1, wherein the probe comprises an infrared spectroscopy to determine oxygen/deoxygenate hemoglobin saturation levels in the user.

9. The system according to claim 1, wherein the probe comprises an impedance/conductance measurement device to determine a change in sweating ability.

10. The system according to claim 1, further comprises a tracking device as part of at least one of the apparatus and the probe to monitor a location of the apparatus.

11. The system according to claim 10, further comprises a GPS system to locate the tracking device.

12. The system according to claim 1, wherein the power source is rechargeable.

13. The system according to claim 1, wherein the power source comprises a wire in a loop configuration to allow inductive charging of the probe.

14. The system according to claim 1, further comprises a warning device that provides information when a change in the environmental condition exceeds a predetermined limit.

15. The system according to claim 14, wherein the warning is device is at least one of part of the apparatus, part of the remote unit, and a second remote unit.

16. The system according to claim 14, wherein the warning device comprises at least one of an audible warning device and a visible warning device.

17. The system according to claim 1, further comprises a storage device to retain information about a change in the user's environmental condition.

18. The system according to claim 17, wherein the storage device is located at least one of as part of the removable apparatus and at a remote unit.

19. The system according to claim 17, wherein data retained in the storage device is compared to actual change in the user's environmental condition.

20. The system according to claim 1, further comprises a storage device that at least one of a charges the power source, sterilizes the removable apparatus, and diagnoses the probe device, when the removable apparatus is in contact with the storage device.

21. A method for detecting at least one of an environmental condition and a physical condition of an entity active in a competitive event wherein a removable apparatus with a probe to detect a change in the environment condition unique to the entity is attached to the entity, the method comprising:
a) detecting a change in at least one of an environmental condition at the removable apparatus;
b) monitoring the change in the at least one environmental condition; and
c) reporting the change in the at least one environmental condition so that the individual may be further evaluated.

22. The method according to claim 21, further comprises communicating a change in the at lest one environmental condition to a remote location.

23. The method according to claim 22, wherein the step of monitoring is performed at a remote location.

24. The method according to claim 21 wherein the step of reporting is accomplished at least at the location of the removable apparatus, the remote location, and a second remote location distance from the first remote location and the removable apparatus.

25. A computer software code implemented through at least one processor for detecting at least one of an environmental condition and a physical condition of an entity active in a competitive event wherein a removable apparatus with a probe to detect a change in the environment condition unique to the entity is attached to the entity, the computer software code comprising:
a) a computer software module for detecting a change in the at least one environmental condition at the removable apparatus;
b) a computer software module for monitoring the change in the at least one environmental condition; and
c) a computer software module for reporting the change in the at least one environmental condition so that the individual may be further evaluated.

26. The computer software code according to claim 25, further comprises a computer software module for communicating a change in the at least one environmental condition to a remote location.
